(19) United States
(12) Patent Application Publication
(10) Pub. No.: US 2007/0161953 A1
(43) Pub. Date: Jul. 12, 2007

(54) Guiding device for the implantation of a needle

(76) Inventors: Mokhtar Chawki, Le Vesinet (FR); Thierry De Catheu, Boulogne Billancourt (FR)

Correspondence Address: Piper Rudnick, P O Box 64087, Chicago, IL, 60664-0807, US

(21) Appl. No.: 10/547,597

(22) PCT Filed: Mar. 3, 2004
(86) PCT No.: PCT/FR04/00502

§ 371 Date: Aug. 22, 2006

(30) Foreign Application Priority Data
Mar. 5, 2003 (FR) 03/02679

(51) Int. Cl. A61M 5/00

(52) U.S. Cl. 604/116; 604/177

(57) ABSTRACT

A guiding device for sub-cutaneous implantation of a veinous puncture needle, comprising a sheath provided with a bore extending according to a main axis enabling the needle to be introduced parallel to said axis, and flexible fins extending radially from said sheath, said fins having a lower surface which can be applied against the skin of a patient and which can assume a rest position wherein the lower surface extends along a plane which is substantially perpendicular to the main axis.

The invention relates to the field of venous puncture.

Venous puncture is performed especially in the context of hemodialysis, in particular in patients with renal insufficiency.

A few notes on the subject of hemodialysis (to which the invention, however, is not limited) would appear necessary at this stage.

Hemodialysis involves removing blood from the patient, circulating the blood through an extracorporeal apparatus called a dialyzer, in which the blood is purified, then returning the blood to the patient. These procedures have to be repeated very frequently, generally at a rate of three sessions a week.

Hemodialysis requires a considerable blood flow (200 to 400 ml per minute). However, a normal vein is unable to provide such a flow rate. It is therefore necessary, in the first instance, to arterialize a vein locally by creating a surgical diversion from an artery to a vein either directly or indirectly (for example by interposition of a natural or synthetic graft).

A subcutaneous vascular access called an arteriovenous fistula is thus created which is identified hereinbelow by the abbreviation AVF and which, after developing to a sufficient diameter (the average period of development being four to six weeks), can be punctured by a qualified person with the aid of a needle.

The puncture procedure traditionally involves inserting one or more needles into the AVF and removing these at the end of the procedure, the AVF being compressed until the bleeding caused by this removal is arrested.

The direct access to the AVF is usually afforded by two independent needles which are introduced tangentially with respect to the AVF (that is to say at a small angle of the order of 20° to 25°). From a semantic point of view, the needle which removes the blood is designated as “artery”, while the needle which returns the blood is designated as “vein”. It is possible to use just one needle serving alternately as “artery” and “vein” by virtue of a Y-junction system.

To ensure a long useful life of the AVF, it is essential that the puncture sites are changed and spaced apart. Conventional needles, because of their orientation, do not easily permit correct spacing of the puncture sites.

This results in repeated use of the same puncture zones, which prevents satisfactory development of the AVFs and promotes the onset of tissue fibrosis of the vessel wall. This tissue fibrosis results in the formation of pseudo-aneurysms and therefore increases the risk of hemorrhagic rupture of the AVF.

The inventors have also found that traditional puncture procedures have the following disadvantages.

First, when the AVF is short or when it is the focus of stenosis, the puncture procedure is awkward because the tangential approach may cause transfixing, this resulting in extravasation of blood fluid into the subcutaneous tissues.

Secondly, in the event of collapse or when the AVF is short, the vessel wall has a tendency to adhere to the needle which is substantially parallel to it and to obstruct the end of said needle, and this promotes the phenomena of hypercoagulability.

Thirdly, the modus operandi of traditional puncturing is rather awkward and requires a relatively long period of training on the part of the person involved (this person can be the patient himself, if he wishes to retain a certain degree of autonomy).

Finally, traditional puncturing with needles implanted in short AVFs entails a high risk of communication between the needles. This phenomenon, called recirculation, reduces the effectiveness of the extracorporeal blood treatment.

The invention aims in particular to overcome the aforementioned disadvantages by proposing a guiding device for subcutaneous implantation of a venous puncture needle which is reliable, practical and safe.

To this end, and according to a first aspect, the invention proposes a guiding device for subcutaneous implantation of a venous puncture needle, this device comprising:
a sheath provided with a bore extending along a main axis in order to permit introduction of the needle parallel to this axis, and
flexible fins extending radially from the sheath, said fins having a lower face which can be applied against the skin of a patient, and being able to assume a rest position in which their lower face extends in a plane substantially perpendicular to the main axis.

The venous puncture can thus be performed perpendicular to the patient's skin (and to the vessel wall). The fins, which in particular have a stabilizing function and ensure that the device is held in position, also form an abutment which limits the depth of insertion of the needle during its subcutaneous introduction.

According to a second aspect, the invention proposes a guiding device for subcutaneous implantation of a venous puncture needle, this device comprising:
a sheath through which a hole extends along a main axis,
flexible fins extending radially from said sheath, said fins having a lower face which can be (and is in fact intended to be) applied against the skin of a patient, and being able to assume a rest position in which their lower face extends in a plane substantially perpendicular to the main axis, and
a sleeve provided with a bore extending along the main axis in order to permit introduction of the needle parallel to this axis, this sleeve being fitted in the hole of the sheath between:
   a use position, allowing the needle to protrude from the device from the lower face of the fins, and
   a retracted position, allowing the sheath to cover the needle.

In addition to the aforementioned advantages of the device according to the first aspect of the invention, this device provides safety of use which limits the risks of injury and/or contamination of the persons carrying out the procedure. This is because the needle disappears inside the sheath in the retracted position and is thus inaccessible.

According to a third aspect, the invention proposes an assembly comprising a device such as the one described above in the first or second aspect, a connector piece, and a needle fitted in this connector piece, the connector piece and the needle being fitted jointly in the device along the main axis.

Other features and advantages of the invention will become clear from the following description and by reference to the attached drawings, in which:

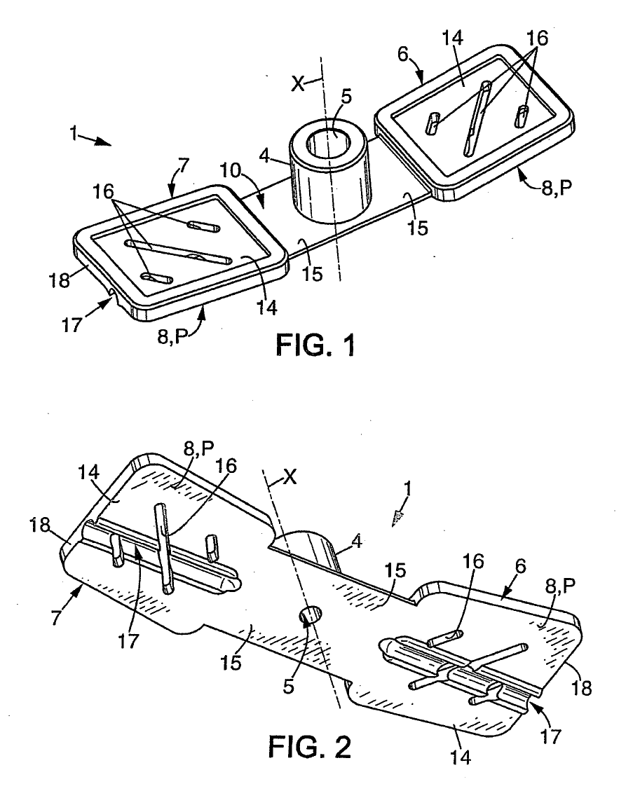

FIG. 1 is a perspective view from above a device according to the invention;

FIG. 2 is a perspective view from below the device in FIG. 1;

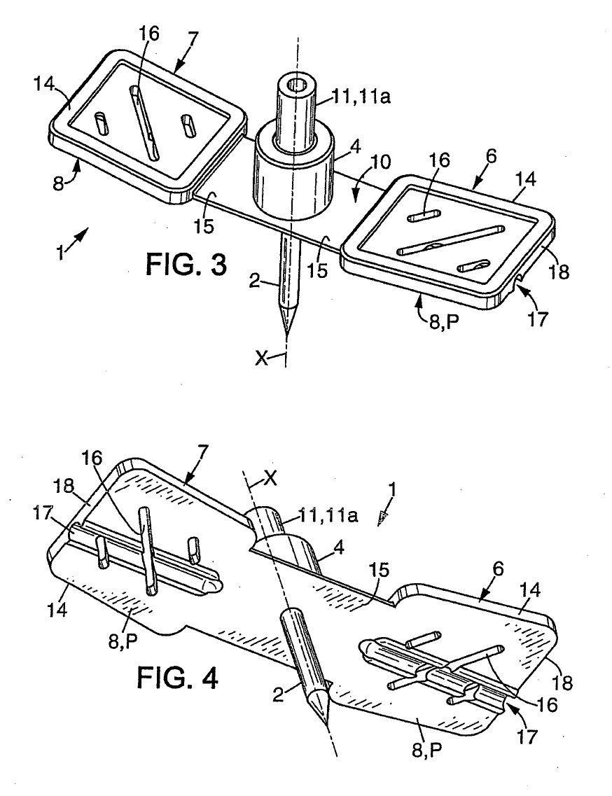

FIG. 3 is a view similar to FIG. 1, illustrating a device according to the invention in which a needle is fitted for the purpose of venous puncture;

FIG. 4 is a bottom view of the device from FIG. 3;

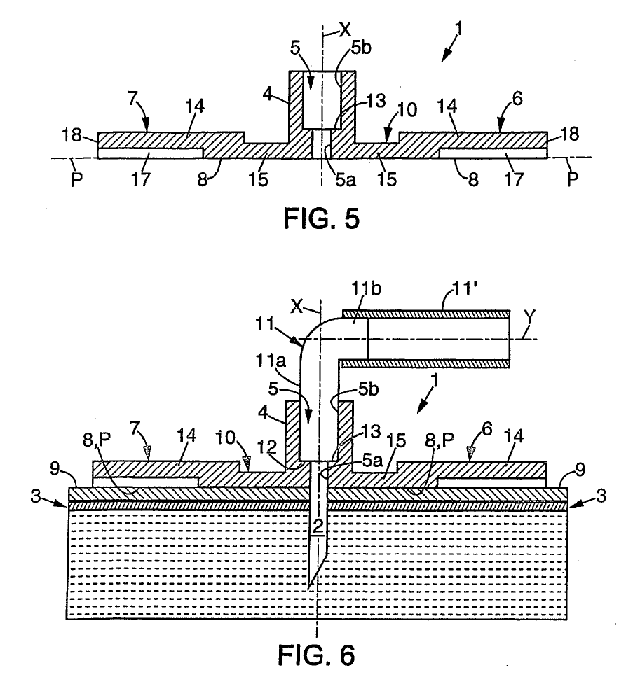

FIG. 5 is a cross-sectional front view of the device from FIGS. 1 and 2;

FIG. 6 is a cross-sectional front view of the device from FIGS. 3 and 4, during use in venous puncture of a patient;

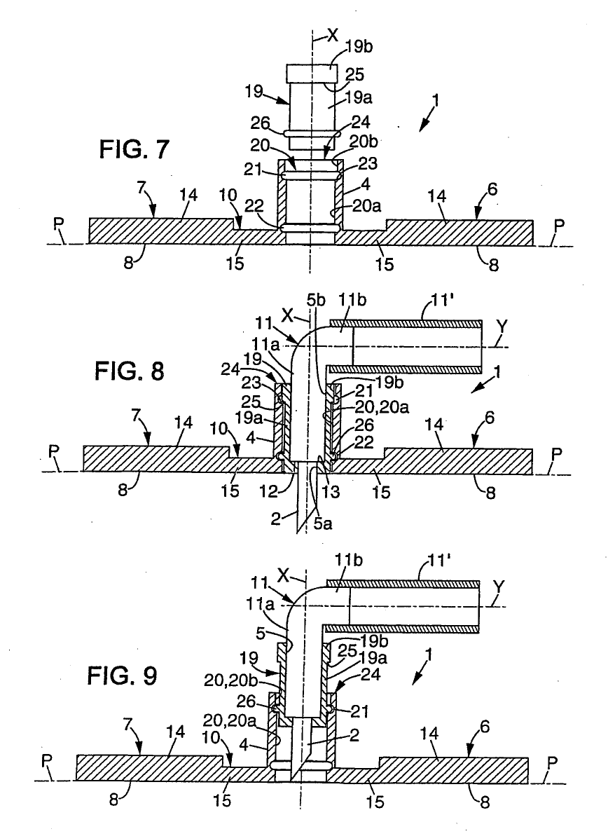

FIG. 7 is an exploded and partially cross-sectional front view of a device according to the invention, according to an alternative embodiment;

FIGS. 8 and 9 are cross-sectional front views illustrating the device from FIG. 7 in which a needle is fitted for venous puncture, in two distinct positions.

The figures show a guiding device 1 for subcutaneous implantation of a needle 2 which is intended for venous puncture, in this case from an arteriovenous fistula (AVF) 3, as is illustrated in FIG. 6.

This device 1 comprises a sheath 4 provided with a cylindrical bore 5 extending along a main axis X and in

which the needle 2 is intended to be introduced, parallel to this axis X.

The device 1 additionally comprises flexible fins 6, 7 which extend radially from the sheath 4.

These fins 6, 7 have a smooth lower face 8 intended to be applied against the skin 9 of a patient, beneath which the AVF 3 extends, and an opposite upper face 10.

In the example shown, the fins 6, 7 are two in number, although their number may be greater, and they extend in the continuation of one another on either side of the sheath 4.

As can be seen from FIGS. 1 and 5, in particular, the sheath 4 is in the form of a hollow cylindrical sleeve which protrudes relative to the fins 6, 7 from their upper face 10.

The fins 6, 7, which are relatively flat, are flexible and are able to assume several positions, each of which positions has a particular function.

Thus, the fins 6, 7 are able to assume:
a rest position, which is illustrated in FIGS. 1 and 6 in particular and in which their lower face 8 extends in a plane P substantially perpendicular to the main axis X so that they can be applied directly against the skin 9 of the patient and are thus able to stabilize the device 1 in a position in which the needle 2 is perpendicular to the skin 9 (FIG. 6),
a grip position, in which the fins 6, 7 are bent toward the main axis X from the direction of their upper face 10, to allow the device 1 to be gripped and removed perpendicular to the skin 9 of the patient when the intervention (for example hemodialysis) is terminated, and
a closure position, in which the fins 6, 7 are bent toward the main axis X from the direction of their lower faces 8 which then touch each other.

In this position, the fins 6, 7 cover the tip of the needle 2 in order to avoid any injury and/or contamination of the person performing the procedure.

Thus, as can be seen from FIG. 5, the device 1 has in cross section, in the rest position, a T shape in which the sheath 4 forms the vertical and the fins 6, 7 form the branches.

The needle 2 is fitted in a rigid connector piece 11 to which a flexible tube 11′ is connected, this flexible tube 11′ itself being connected to an extracorporeal apparatus (not shown) used to purify the withdrawn fluid (for example a dialyzer).

As can be seen from FIG. 6, the connector piece 11 is in this case bent at a right angle: it has a straight main portion 11a which extends along the axis X and terminates in a lower end 12 through which the needle 2 emerges, and a secondary portion 11b (intentionally omitted in FIGS. 3 and 4) which extends along a secondary axis Y perpendicular to the main axis X and on which the tube 11′ is fitted.

As will also be seen from FIG. 6, the bent connector piece 11 allows the tube 11′ to extend parallel to the skin 9 of the patient (in practice the latter's arm), despite the perpendicular orientation of the needle 2, in such a way as to reduce the risks of the device 1 being torn out and to improve patient comfort.

To permit joint introduction of the needle 2 and of the connector piece 11 in the guiding device 1, the bore 5 has a flange 13 separating two portions 5a, 5b of different diameters, against which flange 13 the end 12 of the connector piece 11 will bear (FIG. 6).

According to an embodiment illustrated in the figures, each fin 6, 7 comprises a body 14 in the form of a relatively rigid plate connected to the sheath 4 by a central portion 15 of less thickness and able to permit simple bending of the fin 6, 7.

As will be seen from FIGS. 1 to 4, the fin bodies 14 are provided with slits 16 to allow the patient's skin 9 to breathe during the procedure.

In addition, as will be seen from FIGS. 2 and 4 in particular, each fin 6, 7 is provided with a groove 17 which is hollowed out in its lower face 8 and which extends as far as a free edge 18 of the fin 6, 7 opposite the main axis X.

This groove 18 has a profile in cross section complementing that of the needle 2 (generally circular), the two grooves 18 together forming a protective seat in which the needle 2 is received when the fins 6, 7 are in the closure position, in order to limit the risks of injury and/or contamination of the person performing the procedure, when separating the device 1 from the patient's skin 9 at the end of the procedure.

According to one embodiment illustrated in FIGS. 1 through 6, the device 1 is in the form of a single part; it can be produced by simple molding of a plastic material.

Alternatively, and as is illustrated in FIGS. 7 through 9, the device 1 can comprise an attached movable part.

This variation, in which the reference numbers already employed above are used once again to designate the same elements, differs from the above one in terms of the addition of an attached sleeve 19 which is intended to further improve the safety of the device 1.

The sheath 4 is provided with a hole 20 with main axis X, while the bore 5 through which the needle 2 is introduced is not formed directly in the sheath 4 but in the sleeve 19, which itself is fitted slidably in this hole 20, parallel to the main axis X.

When the sleeve 19 is fitted in the sheath 4, its bore 5, which as has already been stated has a flange 13 to permit joint introduction of the connector piece 11 and of the needle 2 in abutment, extends coaxially with respect to the hole 20, that is to say on the main axis X.

As will be seen from FIG. 7, the hole 20 in the sheath 4 has, about its perimeter, two circumferential grooves spaced apart from one another, namely an upper groove 21 and a lower groove 22 which are both centered on the main axis X.

In addition, the hole 20 has two portions of different diameters separated by a flange, namely a lower portion 20a, which extends between the upper groove 21 and the lower face 8, and an upper portion 20b of greater diameter which extends between the upper groove 21 and an upper end 24 of the sheath 4, opposite from the lower face 8.

With regards to the sleeve 19, it also has two portions of different diameters, namely a lower portion 19a and an upper portion 19b which are separated by a shoulder 25, the lower portion 19a having a circumferential rib 26 about its perimeter complementing the grooves 21, 22.

Thus, the sleeve 19 is mounted slidably in the sheath 4 between:
a use position, illustrated in FIG. 8, in which the rib 26 is in engagement with the lower groove 22, the shoulder 25 being in abutment against the flange 23 in order to prevent the sleeve 19 from sliding relative to the sheath 4 beyond this position in the direction of the lower face 8, and
a retracted position illustrated in FIG. 9, in which the sleeve 19 protrudes from the sheath 4 at its upper end 24, the rib 26 this time being in engagement with the upper groove 21.

In the use position, the needle 2 protrudes from the device 1 from the lower face 8 of the fins 6, 7, in order to permit venous puncture. The force with which the sleeve 19 snaps into the sheath 4 is sufficient to ensure that the resistance exerted by the skin 9 to the introduction of the needle 2 does not cause the sleeve 19 to move accidentally into its retracted position.

In the retracted position, the needle 2 disappears in the sheath 4 which then covers it in order to prevent needlestick injuries and/or contamination. Of course, it is necessary (as will be evident to the skilled person) to choose the relative dimensions of the device 1 and of the needle 2 in such a way that a sufficient length of the needle 2 extends from the device 1 in the use position to permit easy puncturing, while at the same time ensuring a sufficient travel of the sleeve 19 to guarantee total coverage of the needle 2 in the retracted position.

The assembly of the device 1 is very simple. First, the sleeve 19 is introduced into the sheath 4 from the upper end 24, until the rib 26 snaps into the lower groove 22. This snap fit is permitted by the elastic deformation of the material from which the device 1 is made.

The needle 2 is then introduced, from the same side, into the sleeve 19 until the end 12 of the connector piece 11 comes into abutment against the flange 13 provided in the bore 5. The device 1 is then ready for use.

To move the sleeve 19 from its use position to its retracted position at the end of the procedure, it suffices to pull the connector piece 11 parallel to the axis X while firmly holding the fins 6, 7. The elastic deformation of the sheath 4 then allows the rib 26 to escape from the lower rib 22 so as to sit in the upper rib 21.

Other types of mechanisms may be envisaged for fitting the sleeve 19 in the sheath 4 and for moving it from its use position to its retracted position, for example of the quarter turn type, with or without spring.

Irrespective of the embodiment chosen, the device 1 described permits an arrangement of the needle 2 which extends along the main axis X perpendicular to the fins 6, 7 when these are situated in their rest position. It is thus possible to perform a puncturing procedure on the patient perpendicular to the surface of the skin 9.

The fins 6, 7 thus ensure stabilizing of the device 1, which maintains its position (illustrated in FIG. 6) throughout the procedure.

It is possible to still further improve the safety of the device 1 by covering the lower face of the fins 6, 7 with an adhesive substance so that, by adhering to the skin 9 of the patient, the device 1 maintains its position, especially in the event of inadvertent movements.

Venous puncture perpendicular to the skin 9 of the patient, and thus perpendicular to the direction of the flow of blood, permits a more precise and more reliable procedure. The vessel wall is subjected to less trauma during the repeated puncture procedures undergone by the patient. It is thus easier to space the puncture sites apart.

Furthermore, the fins 6, 7 form an abutment beyond which it is no longer possible to drive in the needle 2, and this reduces the risks of transfixing, thereby improving the safety of the patient.

Moreover, the ease of operation of the device 1 allows it to be used even by untrained personnel, and also in particular by the patient himself. The training time needed for this type of procedure is thus shortened.

In addition, the vertical position of the needle 2 (that is to say perpendicular to the flow of blood) decreases the risks of recirculation, that is to say of communication between two needles implanted not far from one another, thereby improving the effectiveness of the procedure.

It should be noted that the embodiments described above must not be considered as constituting any limitation, and alternatives are possible without departing from the scope of the invention.

Thus, instead of providing a pair of fins, it is possible to envision providing the device with a greater number of these, for example four fins distributed in a cross shape about the sheath.

It is also possible to replace the fins with a flexible collar which surrounds the sheath and whose two semicircular portions would each have the functions associated with the fins described above.

As regards the diameter of the needle, this is a matter of choice for the person providing the treatment, depending on each case, and in particular depending on the weakness of the wall and the diameter of the punctured AVF.

However, it should be noted that the device described permits the use of shorter needles, since their implantation is perpendicular to the skin, and thinner needles, since this perpendicular implantation causes virtually no bending stresses on the needle. The use of thinner and shorter needles thus reduces the risk of hematomas, infiltrations and needlestick injuries, and facilitates, on the one hand, the development of the vascular approaches and, on the other hand, frequent changing of the puncture zone.

As regards the diameter of the needle in practice, it may be chosen equal to 16G (Gauge), although greater diameters (14G, 15G) or smaller diameters (17G, 18G) are also conceivable.

In addition, the perpendicular position of the needle does not reduce its aspiration capacity since it is provided at its end with a beveled edge.

Finally, although the device has been described for the guiding of a single needle, it is readily possible to provide two sheaths spaced apart from one another for guiding two parallel needles, namely an artery needle and a vein needle.

1. A guiding device for subcutaneous implantation of a venous puncture needle along a main axis perpendicular to a patient's skin and to the vessel wall, this device comprising a sheath provided with a bore extending along said main axis in order to permit introduction of the needle parallel to this axis, this device being characterized in that the sheath permits engagement of the needle in the bore, and in that it additionally comprises flexible fins extending radially from the sheath, said fins having a lower face which can be applied against the skin of a patient, and being able to assume:
a rest position in which their lower face extends in a plane substantially perpendicular to the main axis, and
a closure position in which the fins extend substantially parallel to the main axis and in which their lower faces touch each other, these fins thus being able to cover the tip of the needle.

2. The device as claimed in claim 1, comprising at least one pair of fins.

3. The device as claimed in claim 2, in which the fins of the same pair extend in the continuation of one another on either side of the sheath.

4. The device as claimed in claim 1, in which the fins are flat and have an upper face opposite the lower face, and in which the sheath protrudes relative to the fins, from their upper face.

5. The device as claimed in claim 1, in which each fin is provided with a groove which is hollowed out in its lower face and which, when the fins are at rest, extends substantially perpendicular to the main axis, two grooves together forming a protective seat for the needle when the fins assume their closure position.

6. The device as claimed in claim 1, in which the lower face of the fins is covered with an adhesive substance.

7. The device as claimed in claim 1, in which said bore is provided with a flange to permit simultaneous introduction of a needle and of a connector piece which, continuing the needle, has a greater diameter than the latter.

8. The device as claimed in claim 1, in which the sheath and the fins form a single-part assembly.

9. A guiding device for subcutaneous implantation of a venous puncture needle along a main axis perpendicular to a patient's skin and to the vessel wall, this device comprising:
a sheath through which a hole extends along said main axis,
flexible fins extending radially from said sheath, said fins having a lower face which can be applied against the skin of a patient, and being able to assume a rest position in which their lower face extends in a plane substantially perpendicular to the main axis, and this device being characterized in that it also comprises a sleeve provided with a bore extending along the main axis in order to permit introduction and engagement of the needle parallel to this axis, this sleeve being fitted in the hole of the sheath between:
a use position, allowing the needle to protrude from the device from the lower face of the fins, and
a retracted position, allowing the sheath to cover the needle.

10. An assembly comprising a device as claimed in claim 9, a connector piece, and a needle fitted in this connector piece, the connector piece and the needle being fitted jointly in the device along the main axis.

11. The assembly as claimed in claim 10, in which the connector piece is bent.

12. An assembly coupling device as claimed in claim 1, a connector piece, and a needle, fitted in this connector piece, the connector piece and the needle being fitted jointly in the device along the main axis.

13. The assembly as claimed in claim 12, in which the connector piece is bent.
